(19) United States
(12) Patent Application Publication
(10) Pub. No.: US 2006/0275849 A1
(43) Pub. Date: Dec. 7, 2006

(54) Monoclonal antibody reagents

(75) Inventors: Dean G. Binger, Saint Louis Park, MN (US); Stephen P. Chan, Minneapolis, MN (US)

Correspondence Address: BECKMAN COULTER, INC., P.O. BOX 169015, MAIL CODE 32-A02, MIAMI, FL, 33116-9015, US

(73) Assignee: Beckman Coulter, Inc., Fullerton, CA (US)

(21) Appl. No.: 11/146,657

(22) Filed: Jun. 7, 2005

(51) Int. Cl. G01N 33/53; C07K 16/40

(52) U.S. Cl. 435/7.92; 530/388.26

(57) ABSTRACT

Methods for purifying monoclonal antibody reagents having increased sensitivity or increased specificity for use in immunoassays for a particular analyte and assays using such antibody reagents. The monoclonal antibody reagents are prepared by serial elution of the monoclonal antibody purification lots at different pHs.

### FIELD OF THE INVENTION

The present invention relates generally to the field of monoclonal antibody reagents, including methods of producing reagents and the use of such monoclonal antibody reagents in immunoassays.

### BACKGROUND OF THE INVENTION

Monoclonal antibodies have become widely used for diagnostic and therapeutic purposes because of their ability to specifically bind to a target analyte of interest. They have been used in immunoassays, diagnostic applications, therapeutic delivery systems, and cellular extractions. As the applications for use of monoclonal antibodies have been identified, and particularly with the use of monoclonal antibodies as part of therapeutic delivery systems, new methods of producing monoclonal antibodies have been developed.

Monoclonal antibodies were first produced in the 1970's through the use of hybridoma technology using a method described by Milstein and Kohler and reported in Nature 256, pages 495-497 (1975). In this method, a mouse, or other suitable animal, is injected with an immunogen. The animal is sacrificed and splenocytes or lymphocytes from the ascites of the animal are fused with a tumor cell line, thus producing hybrid cells or hybridomas. The hybridoma is immortal and capable of producing a genetically coded antibody. The population of hybridomas is screened to isolate individual clones each of which secrete a single antibody species. Currently the desired monoclonal antibodies are grown in either of two ways: by injection into the peritoneal cavity of a suitably prepared mouse (the in vivo, or ascites, method) or by in vitro tissue culture. The simplest approach for producing monoclonal antibodies in vitro is to grow the hybridoma cultures in batches and purify the desired monoclonal antibody from the culture medium. Because the use of serum such as fetal bovine serum in tissue-culture media contains immunoglobulins produced by the animal from which the serum was obtained which resulted in contamination, a number of companies have developed serum-free media formulations to support the growth of hybridoma cell lines.

Immunoglobulins naturally produced in the body all include the same basic units. They all have a four chain structure composed of two identical light chains and two identical heavy chains. Both the heavy and light chain include two regions based on variability in the amino acid sequences; a variable region and a constant region. Immunoglobulins can be divided into five different classes, based on differences in the amino acid sequences in the constant region of the heavy chains. There are five major heavy chain classes designated alpha, gamma, delta, epsilon and mu, and define the corresponding immunoglobulin classes IgA, IgG, IgD, IgE, and IgM, respectively. Some classes are divided into subclasses based on small differences in the amino acid sequences in the constant region of the heavy chains. Four subclasses of IgG, IgG1, IgG2, IgG3 and IgG4, and two subclasses of IgA, IgA1 and IgA2 have been identified.

In addition to the class and subclasses defining the constant heavy chain region, the light chains can be divided into subtypes based on differences in the amino acid sequences in the constant region of the light chain. Characterization of a particular monoclonal antibody into its class and subclass serves as a means to identify the type of monoclonal antibody being produced by the hybridoma. This knowledge may aid in the creation of a purification scheme for a monoclonal antibody based on the particular physical and biochemical properties of its subclass.

Monoclonal or polyclonal antibodies are typically used in immunoassays when an assay must be very sensitive because the amount of analyte in a sample being detected or quantified is in a very low concentration. However, the use of monoclonal or polyclonal antibodies may result in non-specific binding of the antibodies to components present in the patient samples. The presence of such components can interfere with the immunoassay, resulting in false-positive or false-negative results. For example, endogenous immunoglobulins or complement proteins present in the sample may react with the monoclonal or polyclonal antibodies non-specifically. The presence in a patient's serum or plasma of anti-animal antibodies directed against immunoglobulins in general, known as heterophilic antibodies, can potentially produce erroneous results in any immunoassay format using monoclonal or polyclonal antibodies.

In two-site sandwich immunoassays, using two antibodies each directed to a different epitope on an analyte molecule, interference caused by heterophilic antibodies has been recognized as a source of false results. In certain two-site sandwich assays, such as assays for human cardiac troponin I, false-positive results have been frequently reported due to heterophilic antibodies. Heterophilic blocking agents result in reduced false-positives in immunoassays for some samples but not all. Improving the specificity and sensitivity of immunoassays, including two-site sandwich immunoassays, can further reduce the occurrence of false results.

### SUMMARY OF THE INVENTION

Monoclonal antibodies of a particular class and subclass have been produced using the ascites of a mammal and in vitro techniques by growing hybridomas in culture. Monoclonal antibodies of a desired subclass of a class of immunoglobulins have been purified using a variety of methods including the use of affinity chromatography using differences in the affinity to protein A or other ligands. More recently, isoelectric focusing has been used in connection with other separation methods to identify differences in populations of monoclonal antibodies to an analyte of a particular subclass due to differences in charge. Other physical differences may also exist among immunoglobulins separable from a single subclass due to variations in culture conditions. Within highly purified monoclonal immunoglobulin preparations, a plurality of different subpopulations of immunoglobulin molecules separable from within the monoclonal immunoglobulin preparation by varying the elution conditions as a result of slight differences in the physical properties among the different subpopulation. One such slight difference observed among different subpopulations of immunoglobulins separable from a single immunoglobulin subclass or isotype is a difference in apparent charge. For purposes of this disclosure, each such different immunoglobulin molecule or group of immunoglobulin molecules having an identifiable physical property that differs slightly from the same identifiable physical property of other different immunoglobulins separable from an immunoglobulin preparation including immunoglobulins from a single subclass or isotype and which bind to the analyte shall be referred to as an “isosubclass.”

For example, pure IgG subclass preparations have been resolved into individual isoforms. Partitioning of these subclass isoform populations into discrete collective subgroups may be referred to as subclass isoform editing, which may include more than one and less than all of the isoforms present. Subclass isoform editing leads to production of the “isosubclass” populations referred to herein. Each immunoglobulin molecule in the separable groups of immunoglobulin molecules or “isosubclass” has the same amino acid sequence and binds to the analyte used to produce the heterogeneous immunoglobulin preparation. The use of “subclass” in the term “isosubclass” is not limited to differences associated with differences in the heavy chains of the immunoglobulin molecule but also includes differences in physical properties associated with differences in other immunoglobulin components of the molecule.

In another aspect, isosubclasses may be fractionated using difference in the N-linked oligosaccharide profiles. Without being bound to this explanation, the inventors believe that the physical differences among isosubclasses are a result of post-translation modifications and such physical properties may be varied by culture conditions. For example, physical differences in charge among isosubclasses are presumably due to charge masking of specific immunoglobulin epitopes which then incur differences in folding or apparent iso-electric charge. The modifications may be related to altered glycation.

The inventors have found that such physical differences among isosubclasses result in differential functional performance of different subpopulations of isosubclasses in immunoassays and other applications. In an immunoassay, an isosubclass with weak affinity for a target antigen may be more likely negatively impact sensitivity. In an immunoassay, an isosubclass with a more homogeneous glycation pattern may reduce binding with anti-animal immunoglobulins, thus, resulting in reduced false-positive results.

This invention relates to the recognition that different isosubclasses of a subclass of monoclonal antibody preparations obtained from in vivo produced ascites raw material or from antibodies produced using in vitro techniques have physical differences that cause such differential functional performance. In one embodiment, the invention relates to a monoclonal antibody reagent that includes one or more isosubclass obtained from a heterogeneous monoclonal antibody preparation that includes a subpopulation of isosubclasses. In another embodiment, where a monoclonal antibody reagent that binds specifically to an analyte is used in an assay to detect the presence or amount of the analyte and wherein the monoclonal antibody reagent comprises one or more subpopulations of isosubclasses. Each of such subpopulation includes one or more isosubclasses separable from a heterogeneous monoclonal antibody preparation, wherein the monoclonal antibody currently purified from such heterogeneous monoclonal antibody preparation comprises a population of unseparated isosubclasses. As used herein, the term “heterogeneous monoclonal antibody” refers to the entire population of isosubclasses present in a purified monoclonal antibody preparation and wherein such monoclonal antibody binds. The subpopulation of isosubclasses is chosen so that the specificity and/or sensitivity of the assay for the analyte is improved over the specificity and/or sensitivity of the assay when the heterogeneous monoclonal antibody is used.

In one aspect of the invention, methods to separate a subpopulation of one or more isosubclasses from a heterogeneous monoclonal antibody are described, wherein the heterogeneous monoclonal antibody is applied to a separation column of a selective matrix to which the monoclonal antibody will bind, followed by elutions under varying conditions to separate out subpopulations of isosubclasses from the population of unseparated isosubclasses making up the heterogeneous monoclonal antibody. The elution conditions will vary depending upon desired physical differences among the subpopulations of isosubclasses. For instance, in one embodiment, a stepwise process of performing a chromotofocusing elution at different pHs resulted in the production of a subpopulation of separated isosubclass from which one or more undesired isosubclasses present in the population of isosubclasses in the heterogeneous monoclonal antibody are excluded, and/or the inclusion of one or more desired isosubclasses in a subpopulation of separated isosubclasses, wherein the separated subpopulation includes less than all of the isosubclasses present in the population of isosubclasses in the heterogeneous monoclonal antibody.

In yet another embodiment, a monoclonal antibody reagent that specifically binds to human cardiac troponin I antigen (cTnI) is produced including a subpopulation of isosubclasses wherein one or more isosubclasses in the population of isosubclasses present in the heterogeneous monoclonal antibody from which the subpopulation is obtained is not included monoclonal antibody reagent. In one embodiment, the monoclonal antibody is prepared by binding a monoclonal antibody preparation to an affinity resin under high pH conditions, followed by a pre-elution fractionation at a predetermined lower pH to selectively obtain a subpopulations of isosubclasses, wherein one or more isosubclasses from the population of isosubclasses in the heterogeneous monoclonal antibody are excluded. The final monoclonal antibody reagent was then eluted at a predetermined low pH that differs from the pH used to selectively elute a homogeneous antibody fraction that includes a desired subpopulation of isosubclasses. A variety of binding and elution conditions can be used to produce the desired subpopulation of isosubclasses to include in a monoclonal antibody reagent wherein such desired subpopulation is chosen based on the functional performance differences of such subpopulation as compare with the population of all isosubclasses in the heterogeneous monoclonal antibody.

### DETAILED DESCRIPTION OF THE DRAWINGS

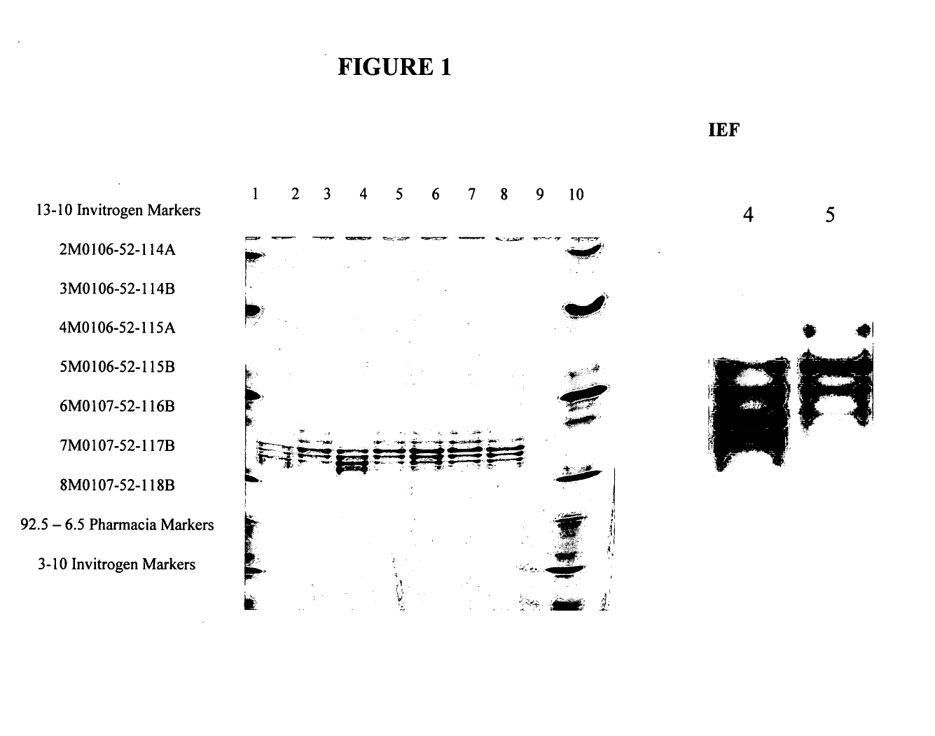

FIG. 1 are illustrations of images of isoelectric focusing gels with elution fractions from purification lots of monoclonal antibodies comparing elution fractions at pH 3.2 to elution fractions at pH 4.5.

### DETAILED DESCRIPTION

In one method of the present invention, ascites or cell culture media including monoclonal immunoglobulin molecules of a single class of immunoglobulins produced using in vivo or in vitro techniques are flowed through an affinity column or other matrix comprising a substrate such as Protein A to which the subclasses of a class of immunoglobulins will differentially bind. The monoclonal immunoglobulin molecules are selected to specifically bind to an analyte of interest. Methods are well known in the art for separating monoclonal antibody preparations of an immunoglobulin class into subclasses or isotypes and purifying fractions of such subclass or isotype. As used herein, the phrase “specifically binds” or “binds specifically” refers to the ability of an immunoglobulin molecule to react immunologically with the analyte of interest.

The bound antibody column is next washed with a wash buffer to remove any impurities from the column. In one aspect of the invention, successive washes with decreasing ionic strength may be used while the pH is held constant.

One or more stepwise elution washes are applied to the bound column of monoclonal antibody preparation. These elutions may be performed using a series of acidic washes at decreasing pH levels. Examples of suitable elution washes include glycine and citric acid. In one embodiment of the invention, the crude monoclonal antibody is bound to the matrix in a high pH buffer. The bound antibody preparation is then washed with stepwise elutions using selective low pH buffers.

The serial elution process takes advantage of the different binding characteristics of the isosubclasses to remove certain isosubclasses from the bound heterogeneous monoclonal antibody population comprising a population of all the isosubclasses produced during the production of the antibody preparation. By eluting the matrix at decreasing pH levels, a distinct subpopulation of one or more isosubclasses is removed with each washing. These subpopulations will contain isosubclasses which exhibit different binding performance. As a result, undesirable isosubclasses may be removed by selective elution to obtain a subpopulation of one or more desirable isosubclass with improved performance.

In one aspect of the invention, the serial elution of the monoclonal antibodies can be used to remove undesirable isosubclasses. After binding, one of more elutions may be performed. The subpopulation of isosubclasses eluted during these elutions would include undesirable isosubclass. One or more subsequent elutions would then be performed under different pH conditions to obtain a desirable subpopulation of isosubclass.

In another aspect the method of this invention can be used to improve sensitivity and/or specificity of an assay for an analyte. Improved sensitivity as used herein, means a lowered background and increased signal to noise ratio obtained with a monoclonal antibody reagent of the invention as compared to the background level and signal to noise ratio obtained in an assay performed under substantially the same conditions using the heterogeneous monoclonal antibody preparation from which the monoclonal antibody reagent was separated. Improved specificity as used herein, means decreased binding of a monoclonal antibody to interfering heterophilic substances in the sample and/or decreased false outcomes in the assay when the monoclonal antibody used in the assay is the monoclonal antibody reagent of the invention over assays performed using a heterogeneous monoclonal antibody preparation as the monoclonal antibody.

Undesirable isosubclasses with low sensitivity and or specificity or with a high likelihood of producing false outcomes may be identified and removed from the heterogeneous monoclonal antibody preparation population through selective chromatofocusing. The resultant subpopulation of isosubclasses is used as a monoclonal antibody reagent in an immunoassay by combining the monoclonal antibody reagent that binds to an analyte with a sample to determine the presence or amount of such analyte in such sample. The monoclonal antibody reagent may be immobilized or is capable of being immobilized upon a solid phase.

The solid phase may be composed, for example, of materials such as glass, paper, polystyrene, polypropylene, polyethylene, dextran, amylases, natural and modified celluloses, polyacrylamides, agaroses, or magnetite. The structural configuration of the solid phase will vary for convenience of use in a particular immunoassay. It may be spherical, as in a bead, cylindrical, as in the inside surface of a test tube or it may be flat, such as a test strip.

Immunoassays are methods for detecting the presence or amount of an analyte in a sample that involves the reaction between at least one antigen (i.e., the analyte) and a least one antibody. An antigen is a substance, such as a protein or carbohydrate, or fraction thereof, which is capable of inducing an immune response when introduced into an animal or human producing immunoglobulins. The site on the antigen to which the antibody binds is referred to as an epitope. Most antigens have multiple and often repeating, binding sites for antibodies. This polyepitopic nature of antigens and the structure of the antibodies (two light chains with epitope binding sites) enable antibody:antigen complexes to be formed in an immunoassay. The presence or amount of analyte in a sample is related to the amount of immune complexes that form through the binding of the antibody to the antigen. In a number of immunoassays, the presence of immune complexes is determined by preparing an indicator reagent comprising a binding protein that specifically binds to the antibody or antigen or immune complex in an amount related to the presence or amount of antigen in the sample wherein the binding protein is labeled with a signal generating compound. “Label”, “labeled” and “labeled conjugate” and the like refer to a conjugate of a binding component or protein with a chemical label such as an enzyme, a fluorescent compound, a radioisotope, a chromophore, or any other detectable chemical specie, the conjugate retaining the capacity to specifically bind to its binding partner.

The improved performance of a monoclonal antibody reagent of the present invention over a heterogeneous monoclonal antibody preparation is particularly useful in assays to detect the presence or amount of human cardiac troponin I (“cTnI”) in a sample. Improving the low-end sensitivity and specificity of an immunoassay for cTnI is challenging because of the absolute absence of cTnI in the blood stream of healthy individuals without myocardial muscle cells damage. In immunoassays using a heterogeneous monoclonal antibody, the incidence of false-positive results attributable in part to the presence of substances such as anti-animal antibodies directed against immunoglobulins known as heterophilic antibodies is of particular importance in this two-site assay.

In another aspect of the invention, the selectively enhanced monoclonal antibody reagent including a subpopulation of desirable isosubclasses of the invention are included in a test kit for detecting the presence or amount of an analyte of interest. “Kit” is used herein to refer to a combination of reagents usually formulated with necessary buffers, salts, and stabilizers, where the reagents are premeasured so as to at least substantially optimize the assay sensitivity. The test kit includes a monoclonal antibody reagent comprises of a subpopulation of isosubclasses from which undesirable isosubclasses have been removed through chromatofocusing elution. The antibody reagent may be immobilized or be capable of being immobilized upon a solid phase such as paramagnetic particles. By removing undesirable isosubclass, the remaining isosubclass may be selected for a specific desired characteristic.

In one embodiment, the subpopulation of isosubclass is chosen such that when the monoclonal antibody reagent is combined with the sample the number of false outcomes (the presence or amount of the analyte of interest is erroneously indicated) is reduced. The test kit may also include an indicator reagent comprising a labeled binding protein that binds specifically to the antibody reagent or the analyte of interest in an amount related to the presence or amount of the analyte of interest in the sample. Examples of an appropriate label include an enzyme, a substrate of an enzyme reaction, a fluorescent label and a chemiluminescent label. One specific label useful for the present invention is alkaline phosphatase.

The following examples are illustrative of the invention and is not intended to limit the scope of the invention as set out in the appended claims.

### EXAMPLE 1

### Chromatofocusing Elution of Anti-cTnI Antibodies Using Glycine

A serum free media was used to grow CTnI19C7 284 cells which produced monoclonal IgG2b anti-cTnI antibodies against human cardiac troponin I, cardiac troponin IC and cardiac troponin ITC complexes. These crude antibodies were immobilized on a Protein A affinity (such as the ProSep A affinity media) resin on a silica matrix at pH 8.6 using a high salt glycine binding buffer. The column of bound antibodies was then washed using successive low salt glycine washes of decreasing ionic strength while the pH was held constant. Elution washes were then performed to separate the isosubclasses. A first elution was performed using 100 mM glycine/HCl at pH 4.5 to obtain one subpopulation of isosubclasses. A lower pH elution was then performed using 100 mM glycine at pH 3.2 to obtain a different subpopulation of isosubclasses. The two elutions were caught on 2 M Tris (pH 8) to immediately neutralize the respective monoclonal antibody pools, minimizing the low pH stress. The elution subpopulations of isosubclasses were compared during subsequent analysis.

The product of various purification lots of IgG2b subclass of monoclonal antibodies to troponin of the 4.5 and 3.2 pH elutions were compared by isoelectric focusing using commercially available isoelectric focusing gels from Invitrogen (Carlsbad, Calif.). Invitrogen protein markers were also used. The results are shown in FIG. 1. Column 1 shows the 3-10 Invitrogen markers. Column 2 shows isosubclasses separated from purification lot M0106-52-114A. Column 3 shows the isosubclasses separated using purification lot M0106-52-114B. Column 4 contains the 4.5 pH eluate with purification lot M0106-52-115A. Column 5 contains the 3.2 pH eluate obtained from purification lot M0106-52-115B. Column 6 shows the separation of purification lot M0107-52-116B. Column 7 shows elution of purification lot M0107-52-117B. Column 8 shows separation of purification lot M0107-52-118B. Column shows separation obtained with 2.5-6.5 protein markers from Pharmacia and Column 10 shows separation with the 3-10 Invitrogen Markers. The separate bands correspond to distinct isosubclasses. The two columns contain three bands in common, indicating that three isosubclasses that were contained in both subpopulation eluates. However, each column (subpopulation) also contains bands (isosubclasses) not found in the other column. These unique bands indicate that certain isosubclasses were selectively removed by the stepwise pH elution process.

As shown in Tables I and IV (see Example 2), the serial elution method of this invention resulted in the 3.2 elution fraction having an improved signal to noise ratio (S1/S0) and increased dose response (S5/S0) as compared to both the control and the 4.5 pH elution fraction. The 4.5 and 3.2 pH eluates were analyzed for assay sensitivity as follows. Each eluted antibody fraction was coupled to paramagnetic particles, a solid phase support for capturing cardiac troponin I (analyte) in a sample. The bound analyte was detected by a second anti-cardiac troponin I monoclonal antibody conjugated to an enzyme. After incubation and a wash step to remove the unbound enzyme, a chemiluminescent substrate was added that reacts with the enzyme label to generate light. The relative light units, RLU, produced were directly proportional to the analyte concentration. The data shown in Table 1 shows the RLU measured on six calibrator levels: 0, 0.3, 1.2, 25 and 100 ng/mL of analyte in S0, S1, S2, S3, S4, and S5, respectively. The signal to noise ratio was calculated from the ratio of RLU of the lowest calibrator level and S0. As shown in Table I, the capture monoclonal antibody purified by sequential elutions of the Protein A bound IgG produced lower S0 RLU and higher RLU responses compared to the conventional single elution buffer at pH 3.2. Both glycine and citrate buffers produced similar results. Purification of the capture monoclonal antibody by sequential elutions with the above buffers at the two different pHs conferred to a cardiac troponin I immunoassay increased sensitivity performance. As shown in FIG. 1, the 3.2 pH eluate contains a population of isosubclasses that differs from the pH 4.5 eluate, and that capture antibody reagent produces better sensitivity than the antibody reagent from the 4.5 or single pH3.2 elution, by excluding less desirable isosubclasses found in the 4.5 pH eluate.

TABLE I
Increased Assay Sensitivity

| Capture | In-vivo | | | | | |
|---|---|---|---|---|---|---|
| Monoclonal | (ascites | In-vitro Bioreactor Produced | | | | |
| Antibody Source | produced) | | Glycine (2 elution | | Citrate 2 elution | |
| ProSepA Affinity | Citrate | Citrate | fractions at 2 pH | | fractions at 2 pH | |
| Chromotography & | pH 3.2 | pH 3.2 | conditions) | | conditions) | |
| Elution Buffer | One step | One step | 1^{st} pH 4.5 | 2^{nd} pH 3.2 | 1^{st} pH 4.5 | 2^{nd} pH 3.2 |
| S0 | 12,786 | 25,092 | 16,972 | 12,494 | 17,147 | 14,120 |
| S1 | 52,332 | 66,049 | 62,170 | 63,727 | 64,664 | 61,681 |
| S2 | 159,492 | 186,003 | 189,454 | 213,529 | 194,755 | 198,986 |
| S3 | 618,246 | 659,327 | 716,231 | 778,546 | 752,056 | 730,228 |
| S4 | 2,955,740 | 3,135,557 | 3,454,145 | 3,881,945 | 3,563,600 | 3,665,210 |
| S5 | 10,707,033 | 11,521,467 | 12,203,750 | 12,982,200 | 13,396,100 | 13,642,850 |
| S1/S0 | 4.1 | 2.6 | 3.7 | 5.1 | 3.8 | 4.4 |
| S5/S0 | 837 | 459 | 719 | 1039 | 781 | 966 |

A cardiac troponin I assay was also performed to assess the specificity of the 4.5 and 3.2 pH glycine dilutions, as shown in Tables II and III. Each eluted antibody fraction was coupled to paramagnetic particles as the solid phase for capturing cardiac troponin I. The bound analyte was detected by a second anti-cardiac troponin I monoclonal antibody conjugated to an enzyme. Monoclonal antibodies produced by ascites grown cells and purified by a standard one step elution at pH 3.2 were used as the control. After incubation and a wash step to remove the unbound enzyme from the sample, a chemiluminescent substrate was added. The RLUs were directly proportional to the analyte concentration. The cardiac troponin I values measured in ng/mL of the twelve samples assayed were determined using a stored multipoint calibration curve, such as the calibration curve used with the commercially available AccuTnI assay calibrators and immunoassay kit sold by Beckman Coulter, Inc. (Fullerton, Calif.). All of the twelve samples produced a false-positive troponin value (greater than the 99^{th} percentile of healthy adults, 0.04 ng/mL) using the control antibody reagent. The first sample shown in Tables II and III was a sample from a mouse IgG immunized goat. The remaining samples were heterophilic patient samples collected from healthy subjects without myocardial injury.

The monoclonal antibody used as the detect monoclonal antibody source in the assays was from different sources. The capture monoclonal antibody purified by sequential elutions yielded improved cardiac troponin I assay specificity compared to the conventional single pH elution step method. The cardiac troponin I values determined by the two-step purified capture monoclonal antibody method were shown to be lower in the first six samples and in the goat anti-mouse serum. The results indicate that the stepwise pH elution contributed to improved assay specificity.

TABLE II
Increased Assay Sensitivity: Lower Heterophile Sample Results

| | Ascites produced | In-vitro | Ascites | In-vitro |
|---|---|---|---|---|
| Capture Monoclonal | Lot 1 | Produced Lot 1 | produced Lot 2 | Produced Lot 2 |
| Antibody | Citrate | Glycine | Citrate | Glycine |
| Elution | (1 step) | (2 steps-) | (1 step) | (2 steps) |
| Goat anti-mouse | 10.45 | 0.23 | 10.30 | 0.21 |
| Patient Sample # 1 | 2.05 | 0.06 | 2.54 | 0.33 |
| Patient Sample # 2 | 1.56 | 0.88 | 3.31 | 2.39 |
| Patient Sample # 3 | 0.15 | 0.00 | 0.19 | 0.00 |
| Patient Sample # 4 | 0.03 | 0.02 | 0.04 | 0.03 |
| Patient Sample # 5 | 0.01 | 0.00 | 0.05 | 0.05 |
| Patient Sample # 6 | 0.00 | 0.00 | 0.06 | 0.06 |
| Patient Sample # 7 | 2.63 | 3.23 | 2.74 | 2.27 |
| Patient Sample # 8 | 0.31 | 0.40 | 0.34 | 0.30 |
| Patient Sample # 9 | 0.17 | 0.38 | 0.18 | 0.12 |
| Patient Sample # 10 | 0.13 | 0.11 | 0.15 | 0.13 |
| Patient Sample # 11 | 0.09 | 0.13 | 0.14 | 0.11 |

TABLE III
Increased Assay Sensitivity: Lower Heterophile Sample Results

| | Ascites produced | In-vitro | Ascites produced | In-vitro |
|---|---|---|---|---|
| Capture Monoclonal | Lot 1 | Produced Lot 1 | Lot 2 | Produced Lot 2 |
| Antibody Source | Citrate | Glycine | Citrate | Glycine |
| Elution Buffer | (1 step) | (2 steps) | (1 step) | (2 steps) |
| Goat anti-mouse | 10.01 | 0.23 | 9.89 | 0.21 |
| Patient Sample # 1 | 1.22 | 0.06 | 2.58 | 0.33 |
| Patient Sample # 2 | 1.21 | 0.88 | 2.84 | 2.39 |
| Patient Sample # 3 | 0.11 | 0.00 | 0.14 | 0.00 |
| Patient Sample # 4 | 0.02 | 0.02 | 0.04 | 0.03 |
| Patient Sample # 5 | 0.00 | 0.00 | 0.05 | 0.05 |
| Patient Sample # 6 | 0.00 | 0.00 | 0.05 | 0.06 |
| Patient Sample # 7 | 2.40 | 3.23 | 2.74 | 2.27 |
| Patient Sample # 8 | 0.37 | 0.40 | 0.39 | 0.30 |
| Patient Sample # 9 | 0.17 | 0.38 | 0.19 | 0.12 |
| Patient Sample # 10 | 0.11 | 0.11 | 0.14 | 0.13 |
| Patient Sample # 11 | 0.10 | 0.13 | 0.15 | 0.11 |

The improved specificity shown in Tables II and III were provided by the capture antibody purified in the purification method of the invention and independent of the second monoclonal antibody used as the detection antibody.

### EXAMPLE 2

### Chromatofocusing Elution of Anti-cTnI Antibodies using Glycine

Monoclonal antibodies were produced, bound to an affinity resin, and washed, as in Example 1. The bound antibodies were then subjected to stepwise elution washes using glycine at pH 4.5 and then pH 3.2.

As above in Example 1, each eluted antibody fraction was coupled to paramagnetic particles as a solid phase support for capturing cardiac troponin I present in the sample. The bound analyte was detected by a second anti-cardiac troponin I monoclonal antibody conjugated to an enzyme, alkaline phosphatase. The RLUs were measured using six calibrator levels: 0.0, 0.3, 1.2, 25 and 100 ng/mL of analyte.

TABLE IV
Increased Assay Sensitivity

| Capture | | | | |
|---|---|---|---|---|
| Mono- | | Lot 1 | Lot 2 | Lot 2 |
| clonal | Lot 1 | (in-vitro | (ascites | (in-vitro |
| Antibody | (ascites produced) | produced) | produced) | produced) |
| Elution | Citrate | Glycine | Citrate | Glycine |
| Buffer | (1 step) | (2 steps-) | (1 step) | (2 steps) |
| S0 | 10,754 | 11,283 | 9,446 | 9,697 |
| S1 | 53,510 | 85,090 | 41,480 | 48,343 |
| S2 | 192,799 | 291,577 | 142,482 | 164,213 |
| S3 | 731,111 | 1,245,390 | 563,641 | 659,608 |
| S4 | 3,254,940 | 5,421,260 | 2,622,875 | 3,210,025 |
| S5 | 10,946,400 | 16,496,350 | 9,478,720 | 10,862,800 |
| S1/S0 | 5.0 | 7.5 | 4.4 | 5.0 |
| S5/S0 | 1018 | 1462 | 1003 | 1120 |

As in Example 1, the capture monoclonal antibody purified by sequential elutions yielded improved cardiac troponin I assay sensitivity compared to the conventional single pH elution step method.

While preferred embodiments of the present invention have been described, it should be understood that various changes, adaptations and modifications may be made therein without departing from the spirit of the invention and the scope of the appended claims.

What is claimed is:

1. An immunoassay for determining the presence or amount of an analyte in a sample, comprising:
providing a monoclonal antibody reagent, wherein said monoclonal antibody reagent comprises a subpopulation of one or more different isosubclasses each of which recognizes and binds specifically to the analyte and wherein such subpopulation includes less than all of the different isosubclasses present in a population of isosubclasses in a heterogeneous monoclonal antibody from which the subpopulation is derived;
combining the monoclonal antibody reagent with the sample for a time sufficient for the monoclonal antibody reagent to bind to analyte in the sample and wherein the immunoassay performed with the monoclonal antibody reagent demonstrates better specificity or sensitivity than an immunoassay conducted using the heterogeneous monoclonal antibody.

2. The immunoassay of claim 1 wherein the monoclonal antibody reagent is immobilized or is capable of being immobilized upon a solid phase.

3. The immunoassay of claim 2 further comprising combining with the sample and monoclonal antibody reagent an indicator reagent comprising a binding protein that binds specifically to the monoclonal antibody reagent or analyte in an amount related to the presence or amount of analyte in the sample.

4. The immunoassay of claim 3, wherein the monoclonal antibody reagent binds specifically to an epitopic site of said analyte that is different from the epitopic site to which the binding protein of the indicator reagent binds.

5. The immunoassay of claim 1 wherein the monoclonal antibody reagent is labeled and the presence or amount of analyte present in the sample is determined by measuring the amount of monoclonal antibody reagent is bound to the analyte.

6. The immunoassay of claim 2, wherein the analyte is troponin.

7. The immunoassay of claim 6 wherein the analyte is cardiac troponin I, cardiac troponin IC or a cardiac troponin ITC complex.

8. The immunoassay of claim 1 wherein the monoclonal antibody reagent is obtained using more than one elutions of a purified monoclonal heterogeneous antibody, wherein a first elution is conducted using a buffer having a first pH and a second elution is conducted using a buffer at a second pH.

9. The immunoassay of claim 8, wherein the first pH is approximately 4.5 and the second pH is approximately 3.2.

10. The immunoassay of claim 7, wherein the monoclonal antibody reagent is an IgG2b anti-cardiac troponin I antibody separated into a subpopulation of isosubclasses.

11. A test kit for detecting the presence or amount of an analyte in a sample, comprising:
a monoclonal antibody reagent, wherein said monoclonal antibody reagent recognizes and binds to the analyte; and is either immobilized upon a solid phase or is labeled with a detectable label, if the monoclonal antibody reagent is immobilized upon a solid phase, the kit further comprises an indicator reagent comprising a labeled binding protein that binds specifically to the antibody reagent or analyte in an amount related to the presence or amount of analyte in the sample or if the monoclonal antibody reagent is labeled with a detectable label, the kit further comprises a capture binding protein bound to a solid phase the capture binding protein being capable of binding to either the monoclonal antibody reagent or analyte in an amount related to the presence or amount of analyte in the sample, and
wherein the monoclonal antibody reagent comprises a subpopulation of one or more isosubclasses each of which recognizes and binds specifically to the analyte and wherein such subpopulation includes less than all of the different isosubclasses present in a population of isosubclasses in a heterogeneous monoclonal antibody from which the subpopulation is derived; and wherein the sensitivity and specificity of the immunoassay using the monoclonal antibody reagent is improved over the sensitivity or specificity of the immunoassay performed using the heterogeneous monoclonal antibody.

12. The test kit of claim 11 wherein said label is selected from the group consisting of an enzyme, a substrate of an enzyme reaction, a fluorescent label and a chemiluminescent label.

13. The test kit of claim 11 wherein the label is alkaline phosphatase.

14. The test kit of claim 11 wherein the analyte is troponin.

15. The test kit of claim 12 wherein the analyte is cardiac troponin I, cardiac troponin IC or a cardiac troponin ITC complex.

16. The test kit of claim 11 wherein the monoclonal antibody reagent is obtained using more than one elutions of a purified monoclonal heterogeneous antibody, wherein a first elution is conducted using a buffer having a first pH and a second elution is conducted using a buffer at a second pH

17. The test kit of claim 16, wherein the first pH is approximately 4.5 and the second pH is approximately 3.2.

18. The test kit of claim 14, wherein the monoclonal antibody reagent is an IgG2b anti-cardiac troponin I antibody separated into a subpopulation of isosubclasses.

19. A monoclonal antibody reagent for specific binding to a target comprising a subpopulation of desired isosubclasses wherein such subpopulation is produced by eliminating one or more isosubclasses from the population of isoclasses present in a heterogeneous monoclonal antibody, wherein said monoclonal antibody reagent recognizes and binds to the target with better specificity than a heterogeneous monoclonal antibody.

20. The monoclonal antibody reagent of claim 19 wherein the monoclonal antibody reagent is obtained using a more than one elutions of a purified monoclonal heterogeneous antibody, wherein a first elution is conducted using a buffer having a first pH and a second elution is conducted using a buffer at a second pH.

21. The monoclonal antibody reagent of claim 19 wherein the target is troponin.

22. A method for producing a monoclonal antibody reagent that binds specifically to a target comprising the steps of:
providing a heterogeneous monoclonal antibody immunoglobulin solution that binds specifically to the target and that comprises a population of isosubclasses;
binding a single subclass of monoclonal antibody immunoglobulins to a selective matrix;
sub-fractionating a subpopulation of isosubclasses of the bound monoclonal antibody immunoglobulin by elution;
selectively removing one or more subpopulations of isosubclasses wherein the subpopulation comprises at least one less isosubclass than the population of the heterogeneous monoclonal antibody immunoglobulin from the matrix; and
selecting one or more subpopulation of isosubclasses for use in a diagnostic assay or to deliver a therapeutic of interest.

23. The method of claim 22 wherein the target is troponin.

24. The method of claim 23 wherein the target is cardiac troponin I, cardiac troponin IC or a cardiac troponin ITC complex.

25. The method of claim 22 wherein the sub-fractionation of the subpopulation of isosubclasses comprises eluting the column using a buffer at a first pH and then eluting the column again using a buffer at a second pH and then collecting the elutions.

26. The method of claim 25, wherein the first pH is approximately 4.5 and the second pH is approximately 3.2.

27. The method of claim 23, wherein the monoclonal antibody reagent is an IgG2b anti-cardiac troponin I antibody separated into a subpopulation of isosubclasses.
